(19) United States
(12) Patent Application Publication
(10) Pub. No.: US 2026/0240548 A1
(43) Pub. Date: Aug. 20, 2026

(54) METHOD AND APPARATUS FOR DETERMINING LIMB OCCLUSION

(71) Applicant: Smart Tools Plus, LLC, Strongsville, OH (US)

(72) Inventor: Nicholas Colosi, Strongsville, OH (US)

(21) Appl. No.: 19/641,109

(22) Filed: Apr. 7, 2026

Related U.S. Application Data
(63) Continuation of application No. 18/233,206, filed on Aug. 11, 2023.
(60) Provisional application No. 63/397,631, filed on Aug. 12, 2022.

(51) Int. Cl. A61B 17/135; A61B 17/00; A61B 90/00

(52) U.S. Cl. CPC A61B 17/135; A61B 2017/00022; A61B 2090/064

(57) ABSTRACT

An apparatus, method, and system that can continuously inflate a cuff to LOP when inflating the cuff that is positioned on a limb of a person or animal without having to terminate operation of the pump to detect if blood is flowing through a blood vessel and past the cuff.

### PRIORITY

The present disclosure is a continuation of U.S. patent application Ser. No. 18/233,206 filed Aug. 11, 2023, which in turn claims priority to U.S. Provisional Application Ser. No. 63/397,631 filed Aug. 12, 2022, which are all fully incorporated herein by reference.

### FIELD OF THE DISCLOSURE

The present disclosure relates to blood flow restriction systems, specifically to a blood flow restriction system that can inflate a cuff to a limb occlusion pressure (LOP) or some upper operation pressure value (UOPV) when inflating the cuff positioned on a limb of a person or animal, and even more specifically to a blood flow restriction system that can continuously inflate the cuff to LOP or some UOPV when the cuff is positioned on a limb of a person or animal without having to terminate operation of the pump to detect if blood is flowing through a blood vessel and past the cuff.

### BACKGROUND

Blood Flow Restriction Training (BFRT) is becoming a popular tool to improve muscle strength, size, and functional aerobic capacity in shorter amounts of time with less stress on the body than typical training. Practitioners began using BFRT in the treatment and recovery from musculoskeletal injuries or disabilities. Trainers and coaches have used BFRT as an adjunct for a usual training regimen or as a tool to aid recovery. BFRT is the brief and intermittent occlusion or restriction of arterial and venous blood flow performed by applying a tourniquet to the upper or lower extremity. BFRT has been found to augment skeletal muscle adaptation, along with systemic whole body changes and cardiovascular benefits while at rest, with low intensity endurance exercises or low load resistance training. BFRT has been found to be safe when applied with pressures relative to the cuff width and individual limb circumference which is obtained through measuring limb occlusion pressures via a Doppler.

BFRT is done by wrapping a tourniquet around the top portion of one's upper or lower limbs. The wrapping restricts blood flow from the veins of the working muscles to the heart and limits the amount of blood flow to the limbs from the arteries. The restriction results in physiological changes that mimic changes associated with high intensity exercise. The results are gains in muscle size and strength and increases in cardiovascular function at much lower intensities than are usually required for adaptation.

Various types of BFRT cuffs have been developed as described in U.S. Pat. Nos. 6,149,618; 7,413,527; 7,455,630; 8,021,283; 8,182,403; 8,273,114; 8,328,693; 8,366,740; 8,425,426; 8,992,397; 9,301,701; and 10,245,458; US Publication Nos. 2009/0124912; 2015/0150560; 2016/0193491; 2017/0112504; 2017/0224357; 2017/0325825; and 2018/0290005; PCT Publication Nos. WO 2016/087123; WO 2017/149690; and WO 2019/068147; and the Smart Cuff™ devices offered by Smart Tools Plus, LLC (https://www.smarttoolsplus.com/), all of which are incorporated herein by reference.

Diaphragm pumps are commonly utilized to inflate the cuff due to their low cost, low audible noise, and small form factor. However, diaphragm pumps move air in small pulses that create small cyclical pressure spikes in the cuff. These pressure spikes compete with the limb's arterial pressure spike caused by a heartbeat, thereby interfering with the determination of whether LOP has been obtained in a limb during the operation of the pump. Several prior art pump systems for inflating a cuff to minimum LOP or some other pressure below or above minimum LOP are disclosed in U.S. Pat. Nos. 10,646,231; 10,646,232; US Publication Nos. 2010/0324429; 2013/021126933; 2013/0211269; and 2013/0184745; all of which are incorporated herein by reference.

Prior art electronic pump control systems for cuffs were required to briefly terminate the operation of the pump so accurate information about blood flow detection through the blood vessel could be determined. Such stopping and starting of the pump increased the time needed to properly inflate the cuff to a minimum LOP or some other pressure below or above minimum LOP. If a minimum LOP determination was required, additional time was required to make such minimum LOP determination. Such added time for cuff inflation was uncomfortable and inconvenient to some users that have a lower pain threshold.

In view of the current state of the art of inflation systems for cuffs, there is a need for a cuff inflation system that can more rapidly determined when a minimum LOP or some other pressure below or above minimum LOP is obtained in a limb and which does not require the stopping and starting of the pump during the cuff inflation process when determining the minimum LOP or some other pressure below or above minimum LOP.

### SUMMARY OF DISCLOSURE

The present disclosure relates to blood flow restriction systems, specifically to a blood flow restriction system that can inflate a cuff to a limb occlusion pressure (LOP) or some upper operation pressure value (UOPV) when initially inflating the cuff positioned on a limb of a person or animal, and even more specifically to a blood flow restriction system that can continuously inflate the cuff to LOP or some UOPV when the cuff is positioned on a limb of a person or animal without having to terminate operation of the pump to detect if blood is flowing through a blood vessel and past the cuff.

In one non-limiting aspect of the disclosure, there is provided an air delivery unit to inflate a cuff and which is configured to constantly supply air to the cuff while simultaneously sensing and determining the LOP or some UOPV of a limb (e.g., arm, leg, etc.) to which the cuff has been releasably connected. Prior art inflation devices that both sense the cuff pressure through the air supply tube and also use the same air supply tube to sense the presence of blood flow past the inflated cuff require the electric air pump (e.g., diaphragm air pump, etc.) to temporarily terminate operation of air supply to the cuff when sensing and making a determination as to whether the LOP of the limb on which the cuff has been connected has been obtained. This termination of the electric air pump was required in such prior art inflation devices since the air pulsations of the air being fed through the air supply tube and to the cuff during the operation of the electric air pump interfered with sensors in the air delivery device from using the same air supply tube to detect whether blood flow past the inflated cuff did or did not exist during a certain cuff inflation pressure. As such, the electric air pump was temporarily stopped to stop the air pulsations to the cuff and thereby allow the one or more sensors in the inflation device to sense signals traveling from the cuff, through the air supply tube, and to the inflation device. Once the sensors in the inflation device determine that blood flow still exists past the cuff, the electric pump is reactivated to again supply air to the cuff for a certain time period and/or until a certain pressure increase in the cuff is obtained. Thereafter, the electric air pump was again termination, and the sensors in the inflation device again made a determination as to whether blood flow still existed past the cuff. This operation was repeated until the sensors determined that there was no blood flow past the cuff. If the minimum LOP needed to be determined, air would be released and then readded to the cuff and the pump repeatedly started and stopped until the minimum LOP is determined. This multiple starting and stopping of the electric air pump during the inflation of the cuff to identify a LOP increased the time needed to obtain an LOP, which could be uncomfortable to some users. The air delivery unit in accordance with the present disclosure overcomes this problem by positioning a pressure sensor port at a location on the cuff that is spaced from the location where air is fed into the cuff (e.g., air input port of the cuff) by the air delivery unit. The pressure/pulse sensor can be directly located on the cuff or in the housing of the pump. When the pressure/pulse sensor is located in the housing, the cuff includes a pressure sensor port that is located in the cuff, and a pressure sensor port is spaced from the location where air is fed into the cuff by the air delivery unit, and the pressure sensor port allows the pressure/pulse sensor in the housing of the pump to measure/detect pressure and/or pressure pulses in the cuff. As can be appreciated, when the pressure/pulse sensor is located on the cuff, but located remotely from the air bladder of the cuff (e.g., located on and/or attached to the top surface of the cuff, etc.), the cuff can include a pressure sensor port that is located in the cuff and which is spaced from the location that air is fed into the cuff by the air delivery unit, and the pressure sensor port allows the pressure/pulse sensor on the cuff to measure/detect pressure and/or pressure pulses in the cuff. As can be appreciated, the pressure/pulse sensor can be located on the cuff and directly connected to the pressure sensor port. By spacing the pressure sensor port from the air input port into the cuff, the pressure/pulse sensor can obtain pressure readings in the cuff as the cuff is inflated without having to stop the operation of the air pump, thus the electric air pump does not require being terminated during the inflation of the cuff when a LOP is being determined. As such, a faster determination of LOP can be obtained by use of the air delivery unit in accordance with the present disclosure.

In another and/or alternative non-limiting aspect of the disclosure, there is provided an air delivery unit used to inflate a cuff wherein the location of the pressure sensor port on the cuff is located at least 1 cm from the location of the air input port of the cuff. The distance between the pressure sensor port and the air input port on the cuff can be greater than 1 cm (e.g., 1.001-100 cm and all values and ranges therebetween), and typically the distance between the pressure sensor port and the air input port on the cuff is about 2-10 cm. In one non-limiting embodiment, during the inflation of the cuff, pressurized air does not flow from the pressure sensor port and into the cuff. In other words, the pressure sensor port is not used as a fluid passageway to inflate the cuff. In another non-limiting configuration, the pressure sensor port is not used to deflate the cuff.

In another and/or alternative non-limiting aspect of the disclosure, there is provided an air delivery unit used to inflate a cuff wherein the housing of the air delivery device is directly connected to the cuff and is configured to remain connected to the cuff during the use of the cuff by the user. In such an arrangement, long air supply hoses (e.g., air supply hoses greater than 4 inches; 0-4 inches and all values and ranges therebetween) or no air supply hose are not required to be connected and/or disconnected from the cuff during a) the inflation of the cuff, b) the deflation of the cuff, and/or c) the use of the cuff by a user. In one non-limiting embodiment, the housing of the air delivery device overlies the air input port when the housing is connected to the cuff. In another non-limiting embodiment, the housing of the air delivery device overlies both the air input port and the pressure sensor port on the cuff when the housing is connected to the cuff. In another non-limiting embodiment, no flexible hose exists between the housing and the cuff when the housing is connected to the cuff. In another non-limiting embodiment, no hose exists between the housing and the cuff when the housing is connected to the cuff.

In another and/or alternative non-limiting aspect of the disclosure, there is provided an air delivery unit used to inflate a cuff wherein the software used to control the operation of the electric air pump and/or to determine LOP or some UOPV in the cuff can be 1) fully stored and/or processed in one or more storage devices and/or processors in the housing of the air delivery device, 2) fully stored and/or processed in one or more storage devices and/or processors that are located remotely from the housing of the air delivery device (e.g., smart device, smart phone, tablet, computer, server, etc.), or 3) be partially stored and/or processed in one or more storage devices and/or processors in the housing of the air delivery device and also be partially stored and/or processed in one or more storage devices and/or processors that are located remotely from the housing of the air delivery device. In one non-limiting embodiment, when the one or more storage devices and/or processors are located remotely from the housing of the air delivery device, the communication between the housing of the air delivery unit and the one or more storage devices and/or processors located remotely from the housing of the air delivery device can wirelessly communicate and/or communicated by one or more wires. In another non-limiting embodiment, when the one or more storage devices and/or processors are located remotely from the housing of the air delivery device, the communication between the housing of the air delivery unit and the one or more storage devices and/or processors located remotely from the housing of the air delivery device is wireless communication (e.g., Bluetooth, Wi-Fi, IR, etc.). In one specific non-limiting embodiment, the housing includes one or more storage devices and/or processors used to both control the operation of the electric air pump and determine LOP or some UOPV in the cuff. Once the LOP or some UOPV has been determined, such information (e.g., information regarding the value of LOP, information that UOPV has been reached, the pressure in the cuff, etc.) can optionally be transmitted to a remote device.

In another and/or alternative non-limiting aspect of the disclosure, there is provided an air delivery unit used to inflate a cuff wherein the pressure/pulse sensor is an electronic pressure/pulse sensor. The pressure/pulse sensor, and/or the hardware and/or software in the housing or remote device can be configured to a) measure a pressure in the cuff, b) detect pressure spikes in the cuff caused by the air pump of the air delivery device, c) detect pressure spikes in the cuff t caused by arterial pressure spikes caused by a heartbeat as blood flows through the artery and past the inflated cuff, d) differentiate the pressure spikes caused by the air pump during inflation of the cuff and pressure spikes caused by a heartbeat as blood flows through the artery and past the inflated cuff, e) determine the frequency of the detected pressure spikes caused by a heartbeat as blood flows through the artery and past the inflated cuff, f) determine the value of the pressure spikes caused by a heartbeat as blood flows through the artery and past the inflated cuff, g) determine a trend of the pressure spikes caused by a heartbeat as blood flows through the artery and past the inflated cuff as to intensity and/or frequency, h) calculate information about the area of the curve of the pressure spikes caused by a heartbeat as blood flows through the artery and past the inflated cuff, and/or i) determine maximum pulsation amplitude of pressure spikes caused by a heartbeat as blood flows through the artery and past the inflated cuff. As can be appreciated, one or more processors and/or software (located in the housing of the air delivery unit and/or located remotely from the housing of the air delivery unit) that are separate from the pressure/pulse sensor can alternatively be used to partially or fully obtain such information, or be used in combination with the pressure/pulse sensor to partially or fully obtain such information.

In another and/or alternative non-limiting aspect of the disclosure, there is provided an air delivery unit used to inflate a cuff wherein the bladder of the cuff in combination with the separate air input port and pressure sensor port of the cuff that are spaced from one another results in the cuff functioning both as a bladder having a) an air volume that can be inflated and deflated, and b) an internal configuration sufficient to dampen the air vibrations/pulses from the air delivery unit to thereby minimize or eliminate air vibration/pulse effects from measurements/data obtained by the pressure/pulse sensor.

In another and/or alternative non-limiting aspect of the disclosure, there is provided an air delivery unit used to inflate a cuff wherein a) the fill air resistance (FAR) of the fluid connection from the electric air pump to the point where the air exits into the bladder of the cuff, and/or b) the pressure sensor air resistance (PSAR) of the fluid connection between the point air enters the fluid sensor port from the bladder of the cuff to the pressure/pulse sensor is selected to improve the accuracy of measurements and/or the ability of the pressure/pulse sensor to collect data from the cuff as the cuff is inflated. With regard to the FAR, the size, length and/or shape of the fluid path between the electric air pump to the point where the air exits into the bladder of the cuff can be selected to obtain a predetermined air flow resistance value R1. With regard to the PSAR, the size, length, and/or shape of the fluid path between the point where air enters the fluid sensor port from the bladder of the cuff to the pressure/pulse sensor can be selected to obtain a predetermined air flow resistance value R2. The selection of the R1 and/or R2 in combination with the air bladder of the cuff can be used to aid in separating and/or “smoothing out” pulsations in the air flow from the air delivery unit to the cuff, thereby enabling information obtained from the pressure/pulse sensor to be processed to screen out or reduce the effect of the air pulsations caused by the electric air pump during the inflating of the cuff. In one non-limiting embodiment, the resistance value R2 can be used to minimize the resistance to air flow from the cuff to the pressure/pulse sensor. By configuring the resistance value R1 and/or the resistance value R2 to suitably minimize pulsation effects from air flow into and/or out of the cuff, the pressure/pulse sensor can more accurately, efficiently, and/or quickly measure the cuff pressure, detect the limb's arterial pressure spikes, and/or measure the limb's arterial pressure spikes. In another non-limiting embodiment, the resistance value R1 is greater (e.g., 1.5-500% greater and all values and ranges therebetween) than resistance value R2. In one non-limiting configuration, the resistance value R1 is 4-200% greater than resistance value R2. In another non-limiting configuration, a cross-sectional size of the opening of the air input port is less than a cross-sectional size of the fluid sensor port. In one non-limiting configuration, the cross-sectional size of the opening of the air input port is 1.1-100 times less (and all values and ranges therebetween) than the cross-sectional size of the fluid sensor port.

In another and/or alternative non-limiting aspect of the disclosure, there is provided an air delivery unit used to inflate a cuff for determining LOP or some upper operation pressure value (UOPV) that includes the steps of:
1) optionally inflating the cuff to a predetermined pressure that is below LOP or UOPV (e.g. 1-150 mmHg below LOP or UOPV) at a generally constant initial air pump speed, and wherein the initial air pump speed is optionally 40-100% (and all values and ranges therebetween) of a maximum air pump speed;
2) inflating the cuff and measuring the pressure and/or pulses in the cuff as the cuff is inflated (e.g., measurements taken at a rate from 1 per 0.001 seconds to one every 20 seconds and all values and ranges therebetween), and wherein during the inflation of the cuff the air pump speed can a) remain constant during the total time period the cuff is inflated, b) vary during the time of inflation of the cuff, and wherein when the air pump speed is varied, the air pump speed is i) initially maintained at an initial air pump speed until the cuff is at the predetermined pressure below LOP or UOPV (e.g., pressure that is 0.1-50% below LOP or UOPV and all values and ranges therebetween or some preset value (e.g., 10-150 mmHg and all values and ranges therebetween), and then reduced and maintained until the cuff is inflated to the LOP or UOPV, ii) initially maintained until the cuff is at the predetermined pressure below LOP or UOPV (e.g., pressure that is 0.1-50% below LOP or UOPV and all values and ranges therebetween), and then reduced to two or more different reduced air pump speeds until the cuff is inflated to the LOP or UOPV or iii) continually varying the air pump speed as the cuff is inflated;
3) optionally digitally filtering the pressure signal produced by the pressure/pulse sensor (e.g., using one or more filters);
4) optionally saving the data from the pressure/pulse sensor;
5) optionally modifying, integrating, and/or processing the real-time data and/or saved data to filter out and/or account for pulses caused by the air pump during the inflating of the cuff; and
6) determining when LOP or UOPV in the cuff is obtained from the modified and/or processed data (e.g., using a peak detection algorithm and % drop in maximum peak pressure and/or width of pulses, etc.).

In another and/or alternative non-limiting aspect of the disclosure, there is provided one or more pressure/pulse sensors that are configured to a) measure a pressure in the cuff, and/or b) detect pressure spikes in the cuff that are caused by i) arterial pressure spikes caused by a heartbeat as blood flows through the artery and past the inflated cuff, and/or ii) pressure spikes caused by the air delivery unit during the inflation of the cuff. In one non-limiting embodiment, the one or more pressure/pulse sensors can be configured to merely measure a pressure in the cuff and/or to detect the pressure spikes that occur in the cuff. The one or more pressure/pulse sensors can optionally be configured to also provide additional information such as, but not limited to, a) the frequency of the detected pressure spike, b) the value of the pressure spikes, c) a trend of the pressure spikes as to intensity and/or frequency, d) information about the area of the curve of the pressure spikes, e) maximum pulsation amplitude of pressure spikes, f) width of the pressure spikes, and/or g) differentiating between pressure spikes caused by the air delivery unit and pressure spikes caused by blood flow through the artery and past the inflated cuff.

In another and/or alternative non-limiting aspect of the disclosure, there is provided a cuff that can optionally be configured as a pulsation bottle used to dampen (i.e., reduce or eliminate) pressure and flow modulations in the apparatus that can be produced by air pulses from the air pump of the air delivery unit. In one non-limiting embodiment, the cuff can optionally be a “volume bottle” that includes no internal baffles. In another non-limiting embodiment, the cuff can optionally include internal baffles suitably sized, shaped, and positioned to dampen the air pulses into the cuff from the air pump of the air delivery unit as the air pressure in the cuff is detected by the pressure/pulse sensor.

In another and/or alternative non-limiting aspect of the disclosure, there is provided a non-limiting method for determining LOP in the cuff without having to terminate operation of the air pump during the inflation of the cuff, and wherein the method includes the steps of:
Step 1—The air delivery unit pumps fluid (e.g., air, etc.) into the cuff to a predetermined initial pressure (e.g., 20-150 mmHg and all values and ranges therebetween). Such predetermined initial pressure is selected to be below a LOP. During the inflation of the cuff to the predetermined initial pressure, the air delivery unit can optionally operate at about 40-100% (and all values and ranges therebetween) of the maximum air output and/or maximum pump speed to rapidly inflate the cuff to the initial predetermined pressure. Step 1 is an optional step and Step 2 can be the first step.
Step 2—After the optional initial pressurization of the cuff at Step 1, the cuff continues to be inflated. In one non-limiting embodiment, (when Step 1 is used) the inflation rate of the cuff during Step 2 can optionally be less than the inflation rate of the cuff during Step 1; however, this is not required. During the inflation of the cuff during Step 2, the inflation rate can be constant until the cuff reaches LOP, or the inflation rate of the cuff can be variable during the inflation of the cuff, or the inflation rate can be initially constant and then the inflation rate can be varied (e.g., reduce the inflation rate, etc.) as the pressure in the cuff nears the LOP. In one specific arrangement, the inflation rate of the cuff is reduced a) as the pressure in the cuff is at or exceeds a preset pressure (e.g., 50 -150 mmHg and all values and ranges therebetween), and/or b) as the pressure in the cuff nears (e.g., within 5-50% and all values and ranges therebetween) the LOP.

During optional Step 1 and Step 2, or only beginning at Step 2, the one or more pressure/pulse sensors are configured to measure pressure/pulses in the cuff. The rate at which the one or more pressure/pulse sensors measures pressure/pulses in the cuff is non-limiting (e.g., 1-2000 pressure measurements per second and all values and ranges therebetween). The pressure/pulses measurements by the one or more pressure/pulse sensors can optionally be digitally filtered using one or more filters. In one non-limiting embodiment, the pressure/pulse measurements are filtered by one or more filters. In one non-liming specific configuration, the pressure/pulse measurements are filtered by at least two low pass filters that have different cut-off frequencies. The cut-off frequencies of the one or more filters is non-limiting. In one non-limiting embodiment, the first low pass filter, LP1 has a cut-off frequency that is at least 2 times (e.g., 2-500 times and all values and ranges therebetween) different from the second low pass filter, LP2. The filters in combination with the selected R1 and R2 and the bladder of the cuff facilitate in isolating the pulse spike in the cuff caused by the blood flow past the cuff that is caused by a heartbeat. During the pressure/pulse measurements, the pressure/pulse measurements can be recorded, and/or the filtered data can be recorded, processed into a graph, etc. One or more processors and/or hardware and/or software can be used to process the data. In one non-limiting embodiment, the data values from one of the filters is processed (e.g., subtracted, added, integrated, etc.) relative to the data values from another filter to produce a data set that is representative of one or more of the arterial pulses detected by the one or more pressure/pulse sensors.

During optional Step 1 and Step 2, or only beginning at Step 2, the data representative of a plurality of arterial pulses can optionally be further processed by calculating a dynamic linear regression trendline to compensate for the increasing/decreasing pressure trends in the cuff. During the processing of the data obtained by the one or more pressure/pulse sensors, the linear regression trendline can be created at real-time or near real-time. The linear regression trendline can optionally generate a modified data set to center or orient one or more or all of the data sets that are representative of the arterial pulses about a common axis (e.g., 0 axis).

The data sets that are representative of the arterial pulses (whether modified by use of a linear regression trendline or unmodified) can be used to determine when LOP is obtained during the inflation of the cuff without having to terminate operation of the air pump in the air delivery unit. In one non-limiting embodiment, the height/magnitude of the data sets or graphed results that are representative of the arterial pulses, the area of peaks of the data sets or graphed results that are representative of the arterial pulses, and/or the width of the peaks of the data sets or graphed results that are representative of the arterial pulses can be used determine when LOP is obtained during the inflation of the cuff. In one non-limiting embodiment, the area under each of the peaks of the data sets or graphed results that are representative of the arterial pulses is calculated by integrating the pressure points over time for all band filter values (e.g., for only the positive band filter values, for any of the band filer values, etc.). The calculated area under each of the peaks of the data sets or graphed results that are representative of the arterial pulses can then be used to determine if LOP has occurred in the cuff. In one non-limiting embodiment, LOP can be calculated by employing a peak detection algorithm. Each pressure pulse can be represented and/or graphed to show a maximum pressure for each pulse that is representative of the calculated area under each of the peaks of the data sets or graphed results that are representative of the arterial pulses. By utilizing the peak detection algorithm, a drop in the peak value as a percentage of the maximum pressure for the detected/measure pulses can be detected/determined. For example, a drop in the peak value of at least 25% (e.g., 25-100% and all values and ranges therebetween) can be considered to correspond to the LOP of the cuff. The maximum pressure of the detected/measure pulses can be determined as the maximum detected pressure prior to at least one (e.g., 1-20 and all values and ranges therebetween) measured pressure drops. Once a maximum pressure of the detected/measure pulses is determined, then LOP can be determined if a subsequent measured pressure drop of a detected/measure pulses falls below a preset value or cut-off and/or falls below some preset percentage of the maximum pressure. In one non-limiting embodiment, the LOP is determined if a subsequent measured pressure drop of a detected/measure pulses falls below a preset value or cut-off of at least 25% of the value of the maximum pressure. When utilizing the apparatus and system of the present disclosure, a more efficient, precise, and reliable method for determining LOP based on pressure measurements is obtained without the need to stop the operation of the air pump during the detection of pulses in the cuff. Such result is accomplished at least in part by one or more of: the reduction in noise due to the predetermined resistance in the air tubes and/or the pulse bottle configuration of the cuff; the low pass filtering of pressure data, calculations of the peak height, area under the peak, and/or how wide/narrow the detected/measured heartbeat pulses are; the integration of heartbeat curve to calculate pressure peaks; and the determination of a relative drop as a percentage of the maximum peak pressure.

In another and/or alternative non-limiting aspect of the disclosure, there is provided a housing that partially or fully includes the air delivery unit and the pressure/pulse sensor, and wherein the housing at least partially contains a) a motor; b) a pump; c) optionally a power source; d) one or more pressure sensors and/or one or more pulse sensors; e) one or more circuit boards and other electronics; f) one or more processors (e.g., controller, etc.); g) optionally one or more pressure gauges; h) software; i) optionally one or more displays (e.g., LED screen, OLED screen, etc.); j) optionally a power port; k) optionally a data port (e.g., micro USB port, etc.); l) an optional GPS system; m) optional wireless electronics (e.g., transmitter and/or receiver, etc.) to send/receive wireless signals; n) optionally one or more buttons (e.g., power button, home button, power off button, reset button, etc.); o) optionally one or more selection or scroll buttons to enable manual operation/control of the air delivery unit; p) optionally one or more switch(es) to enable manual operation/control of the air delivery unit; q) connection arrangement to permanently or releasably connect the housing to the cuff; r) optionally one or more air ports; s) optionally one or more valves, t) optionally one or more air tubes, u) optionally one or more temperature sensors, v) optionally a gyroscope, and/or w) optionally one or more lights and/or other indicators to indicate a mode, operation, and/or status of the air delivery unit and/or pressure/pulse sensor. The shape and size of the housing is non-limiting.

These and other objects and advantages will become apparent from the discussion of the distinction between the disclosure and the prior art and when considering the non-limiting embodiment illustrated in the accompanying drawings.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIG. 1 is a schematic depiction of a representative apparatus being used on a limb of a user.

FIG. 2 is a schematic depiction of a representative apparatus being used on a limb of a user.

FIG. 3 is a flow diagram for a method of the disclosure.

FIG. 4 is a graph showing data from one pressure pulse gathered by the system of the disclosure.

FIG. 5 is a graph derived from manipulation of the data of the graph of FIG. 4.

FIG. 6 is a graph of one arterial pressure pulse and a calculated line of linear regression.

FIG. 7 is a graph showing adjusted arterial pressure pulse data based on the line of linear regression shown in FIG. 6.

FIG. 8 is a graph showing multiple, sequential arterial pressure pulses.

FIG. 9 is a graph showing an integration of the sequential arterial pressure pulses of FIG. 10.

FIG. 10 is a graph showing the peak pressure levels for the arterial pressure pulses of FIG. 11.

FIG. 11 depicts an example cuff of the present disclosure.

FIGS. 12A-C depicts a three views of a representative system of the disclosure in a packaged configuration.

### DETAILED DESCRIPTION OF NON-LIMITED EMBODIMENTS

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of’ and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of’ and “consisting essentially of’ the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 inches to 10 inches” is inclusive of the endpoints, 2 inches and 10 inches, and all the intermediate values).

The terms “about” and “approximately” can be used to include any numerical value that can vary without changing the basic function of that value. When used with a range, “about” and “approximately” also disclose the range defined by the absolute values of the two endpoints, e g. “about 2 to about 4” also discloses the range “from 2 to 4.” Generally, the terms “about” and “approximately” may refer to plus or minus 10% of the indicated number.

Various non-limiting embodiments of the present disclosure will now be described to provide an overall understanding of the principles of the structure, function, and use of the apparatus, systems and methods disclosed. Those of ordinary skill in the art will understand that apparatus, systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure. Any failure to specifically describe a combination or sub-combination of components should not be understood as an indication that any combination or sub-combination is not possible.

It will be appreciated that modifications to disclosed and described examples, arrangements, configurations, components, elements, apparatuses, devices, systems, methods, etc. can be made and may be desired for a specific application. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

The apparatus, system, and method of the present disclosure relates to and facilitates the sensing of blood pressure in the limb of a human being, e.g., the upper arm.

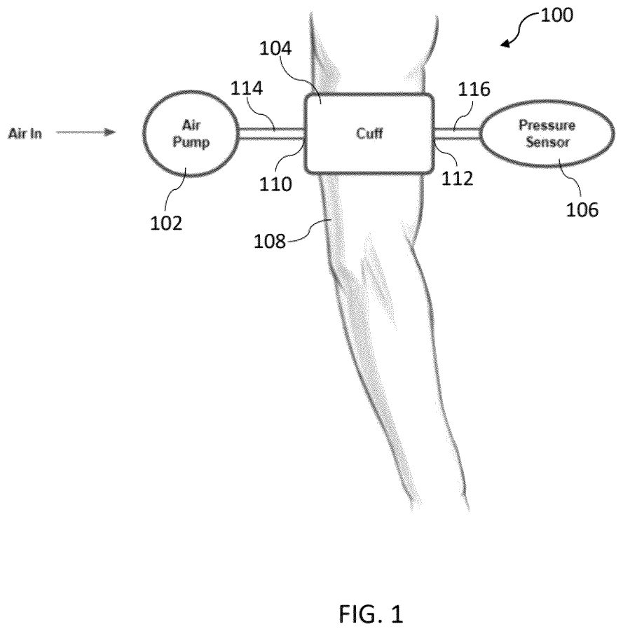

As depicted in FIG. 1, a representative, non-limiting cuff apparatus 100 that includes an air delivery unit 102 (such as an air pump) that can pump air into a cuff 104 configured to wrap round a portion of the limb 108 of a user (e.g., leg, arm, etc.). Air delivery unit 102 can include an electro-mechanical pump; however, other types of motorized air pumps can be used. The air pressure in the cuff can be sensed, analyzed, and/or displayed by a pressure/pulse sensor 106 that is suitably connected to cuff 104. It is to be appreciated that other devices can be utilized to deliver air into cuff 104, such as air from a compressed air source. It is also to be appreciated that other fluids, including gases other than air (e.g., nitrogen) can be pumped into cuff 104.

As illustrated in FIG. 1, two ports (i.e., air input port 110 and fluid sensor port 112 on cuff 104 can be utilized for air communication into/out and/or with cuff 104. Air input port 110 can be configured to facilitate air communication into and/or out of cuff 104 from air delivery unit 102. An input tube 114 (e.g., flexible input tube, etc.) can optionally be used to connect air input port 110 to air delivery unit 102. Alternatively, air delivery unit 102 can be directly connected to cuff 104 and air input port 110, thus potentially eliminating the need for an input tube. Air input port 110 can be used to allow air or some other fluid to enter and/or exit cuff 104 to facilitate in the inflation and/or deflation of cuff 104. In one configuration, there is a housing that is directed connected to the cuff and which housing partially or fully includes air delivery unit 102, and the housing optionally partially or fully overlies air input port 110 when connected to the cuff, and an air passageway that fluidly connects air delivery unit 102 to air input port 110 can a) optionally include a short (e.g., 0.01-4 inch tube and all values and ranges therebetween) flexible or non-flexible tube, or b) be a connector that directly connects air delivery unit 102 to air input port 110.

A fluid sensor port 112 can be configured to facilitate air communication of cuff 104 to a pressure/pulse sensor 106. An output tube 116 (e.g., flexible output tube, etc.) can optionally be used to connect fluid sensor port 112 to pressure/pulse sensor 106. Alternatively, the pressure/pulse sensor can a) be directly connected to cuff 104 and fluid sensor port 112, or b) a housing that includes air delivery unit 102 can also include pressure/pulse sensor 106 can be directly connected to cuff 104 and air input port 110, thus potentially eliminating the need for an output tube. In one configuration, there is a housing that is directed connected to the cuff and partially or fully includes pressure/pulse sensor 106 and optionally also partially or fully includes air delivery unit 102, and the housing optionally partially or fully overlies fluid sensor port 112 and optionally also partially or fully overlies air input port 110 when connected to the cuff, and an air passageway that fluidly connects pressure/pulse sensor 106 to fluid sensor port 112 can a) optionally include a short (e.g., 0.01-4 inch tube and all values and ranges therebetween) flexible or non-flexible tube, or b) be a connector that directly connects pressure/pulse sensor 106 to fluid sensor port 112.

The pressure/pulse sensor 106 can be an electronic pressure/pulse sensor. Pressure/pulse sensor 106 can be configured to a) measure a pressure in the cuff, and/or b) detect pressure spikes in the cuff that are caused by i) arterial pressure spikes caused by a heartbeat as blood flows through the artery and past the inflated cuff, and/or ii) pressure spikes caused by air delivery unit 102 during the inflation of the cuff. Pressure/pulse sensor 106 can be simply configured to merely measure a pressure in the cuff and/or to detect the pressure spikes that occur in the cuff. Pressure/pulse sensor 106 can optionally be configured to also provide additional information such as, but not limited to, a) the frequency of the detected pressure spike, b) the value of the pressure spikes, c) a trend of the pressure spikes as to intensity and/or frequency, d) information about the area of the curve of the pressure spikes, e) maximum pulsation amplitude of pressure spikes, and/or f) differentiate between pressure spikes caused by air delivery unit 102 and pressure spikes caused by blood flow through the artery and past the inflated cuff, etc.

As discussed more fully below, having two ports into cuff 104 affords at least the advantage of allowing cuff 104 to function as a bladder having an air volume and/or internal configuration sufficient to dampen the air vibrations from the air pump of air delivery unit 102 to thereby minimize or eliminate air vibration effects in pressure/pulse sensor 106 that are caused by fluid flow into the cuff caused by air delivery unit 102. Pressure/pulse sensor 106 can be configured to measure the pressure in cuff 104 and/or the limb's arterial pressure spikes without having to terminate operation of air delivery unit 102 during the inflation of cuff 104.

The air pump in air delivery unit 102 can be any suitable air pump. In one non-limiting embodiment, the air pump can “pulse” air in small quantities of relatively high air pressure. The air pulsations from the pump of air delivery unit 102 can enter cuff 104 through air input port 110.

Cuff 104 can optionally be configured as a pulsation bottle. A pulsation bottle is a device used to dampen (i.e., reduce or eliminate) pressure and flow modulations in the apparatus that can be produced by air pulses from the air pump of air delivery unit 102. In another non-limiting embodiment, cuff 104 can optionally be a “volume bottle” that includes no internal baffles. In another non-limiting embodiment, cuff 104 can optionally include internal baffles (not shown) suitably sized, shaped, and positioned to dampen the air pulses into the cuff from the air pump of air delivery unit 102 as the air pressure in the cuff is detected by pressure/pulse sensor 106. In another non-limiting embodiment, the optional internal baffles of cuff 104 can include one or more baffle plates and one or more choke tubes suitably configured to limit pressure pulsation to within an acceptable range (e.g., limit pressure pulsation to within +/−1-10% and all values and ranges therebetween) of the pressure while at the same time limiting pressure drop to an acceptable value (e.g., about 0.1-5% of absolute pressure and all values and ranges therebetween). In one specific non-limiting arrangement, the cuff 104 includes a single chamber inflatable tube or bladder that is absent internal baffles.

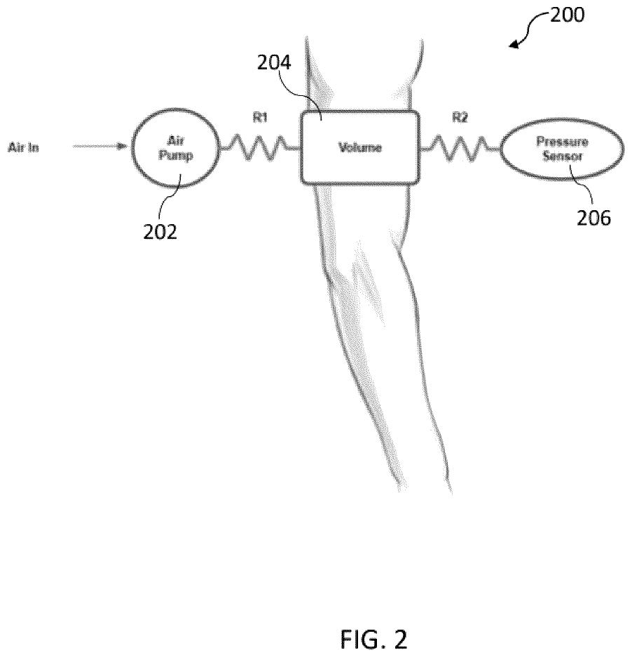

Referring now to FIG. 2, a representative, non-limiting cuff apparatus 200 is depicted schematically. Cuff apparatus 200 can have any or all the components of cuff apparatus 100, described above. In FIG. 2, cuff apparatus 200 is shown denoting a resistance value R1 for fluid flowing from air delivery unit 202 and into cuff 204, and a resistance value R2 for flow of fluid between cuff 204 and pressure/pulse sensor 206. In one non-limiting embodiment, the flow path between air delivery unit 202 and cuff 204 can be suitably sized and shaped to provide a predetermined resistance value R1. Likewise, the flow path between cuff 204 and pressure/pulse sensor 206 can be suitably sized and shaped to provide a predetermined resistance value R2. In another non-limiting embodiment, resistance value R1 can be suitably configured to aid in separating, or “smoothing out” pulsations in the air flow from air delivery unit 202 to cuff 104 and, ultimately, pressure/pulse sensor 206. In another non-limiting embodiment, resistance value R2 can be suitably configured to minimize the resistance to air flow from cuff 204 to pressure/pulse sensor 206. By configuring resistance value R1 and/or resistance value R2 to suitably minimize pulsation effects from air flow into and/or out of cuff 204, pressure/pulse sensor 206 can more accurately, efficiently, and quickly measure the cuff pressure, detect the limb's arterial pressure spikes, and/or measure the limb's arterial pressure spikes. In another non-limiting embodiment, resistance value R1 is greater (e.g., 4-500% greater and all values and ranges therebetween) than resistance value R2. The cross-sectional shape of air input port 110, the maximum and/or minimum cross-sectional size of air input port 110, the length of the fluid connection between air delivery unit 202 and air input port 110, and/or the cross-sectional size and/or shape of the fluid connection between air delivery unit 202 and air input port 110 can be used to obtain the desired resistance value R1. Likewise, the cross-sectional shape of fluid sensor port 112, the maximum and/or minimum cross-sectional size of fluid sensor port 112, the length of the fluid connection between pressure/pulse sensor 206 and fluid sensor port 112, and/or the cross-sectional size and/or shape of the fluid connection between pressure/pulse sensor 206 and fluid sensor port 112 can be used to obtain the desired resistance value R2. In one non-limiting configuration, the cross-sectional shape and/or size of fluid sensor port 112 is greater than the cross-sectional shape and/or size of air input port 110.

By configuring cuff 104, 204 as a pulsation bottle and/or configuring suitable resistance values R1 and R2, cuff apparatus 100, 200 can be figured to be a pneumatic system wherein pressure/pulsation sensor 106, 206 that can more accurately and efficiently measure a) the pressure in cuff 104, 204, and/or b) the limb's arterial blood pressure spikes while the air pump of air delivery unit 102, 202 is on and providing pressure pulses to cuff 104, 204 during the inflation of cuff 104, 204.

Cuff apparatus 100, 200 can be used to determine when limb occlusion pressure (LOP) or some upper operation pressure value (UOPV) is reached while cuff 104, 204 is positioned on the limb of a user and while the air pump of air delivery unit 102, 202 is operating. Such a configuration of cuff apparatus 100, 200 in accordance with the present disclosure is a significant advancement over prior art cuff inflation devices wherein the pump must be temporarily stopped and restarted multiple times during the inflation of the cuff so that information about the limb's arterial blood pressure spikes can be obtained while the pump is stopped.

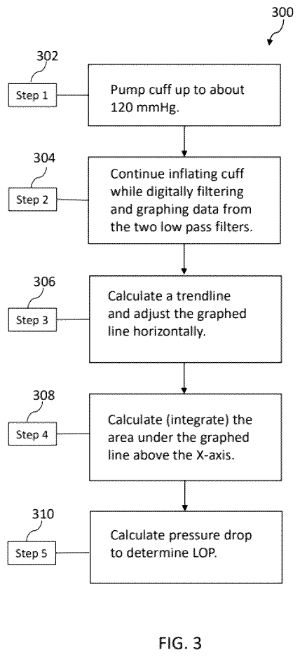

A representative non-limiting embodiment of a method 300 for determining LOP or some UOPV in accordance with the present disclosure is schematically depicted in FIG. 3. The method is described in the context of apparatus 200. In Step 1/302, air delivery unit 202 pumps air into cuff 204 to a pressure in the cuff of about 120 mmHg; however, it will be appreciated that some other initial pressure value can be used (e.g., 0-150 mmHG and all values and ranges therebetween). Such initial pressure is selected to be significantly below a LOP or some UOPV. Generally, air delivery unit 202 operates at about 40-100% (and all values and ranges therebetween) of the maximum air output and/or maximum pump speed, and typically about 60-100% of the maximum air output and/or maximum pump speed so as to rapidly inflate cuff 204 to the initial pressure. As can be appreciated, Step 1 is an optional step and Step 2/304, can be the first step.

After the optional initial pressurization of the cuff at Step 1, cuff 204 continues to be inflated at Step 2/304. In one non-limiting embodiment (when Step 1 is used), the inflation rate of cuff 204 during Step 2 can optionally be less than the inflation rate of the cuff during Step 1. In one non-limiting embodiment, pressure/pulse sensor 206 is configured to measure pressure in the cuff at a rate of 20 pressure measurements per second (50 Hz); however, other rates of measuring pressure can be used (e.g., 1-2000 pressure measurements per second and all values and ranges therebetween). During the inflation of the cuff during Step 2, the inflation rate can be constant until the cuff reaches LOP or some upper operation pressure value (UOPV), or the inflation rate of the cuff can be variable during the inflation of the cuff, or the inflation rate can be initially constant and then varied (e.g., reduce the inflation rate, etc.) as the pressure in the cuff nears the LOP or some UOPV. In one specific arrangement, the inflation rate of the cuff is reduced a) as the pressure in the cuff is at or exceeds a preset pressure (e.g., 50 -150 mmHg and all values and ranges therebetween), and/or b) as the pressure in the cuff nears (e.g., within 5-50% and all values and ranges therebetween) the LOP or some UOPV.

The pressure measurements at Step 2/304, can optionally be digitally filtered using one or more filters. In one non-limiting embodiment, the pressure measurements are filtered by at least two low pass filters, a first low pass filter LP1 and a second low pass filter LP2, with cut-off frequencies of 1 Hz and 10 Hz, respectively. As can be appreciated, one filter can be used or more than two filters can be used. As also can be appreciated, the cut-off frequencies of the one or more filters is non-limiting. In one non-limiting embodiment, first low pass filter LP1 has a cut-off frequency that is at least two times (e.g., 2-500 times and all values and ranges therebetween) different from second low pass filter LP2. The filters in combination with selected R1 and R2 and the bladder of the cuff are used to isolate the pulse spike in the cuff caused by the blood flow past the cuff that is caused by a heartbeat.

First low pass filter LP1 can optionally be a Butterworth™, low pass, second order, 50 Hz sample rate, low corner: 1 Hz, upper corner: 1 Hz. Second low pass filter LP2 can also optionally be a Butterworth™, low pass, second order, 50 Hz sample rate, low corner: 10 Hz, upper corner: 1 Hz. As can be appreciated, other types of filters can be used.

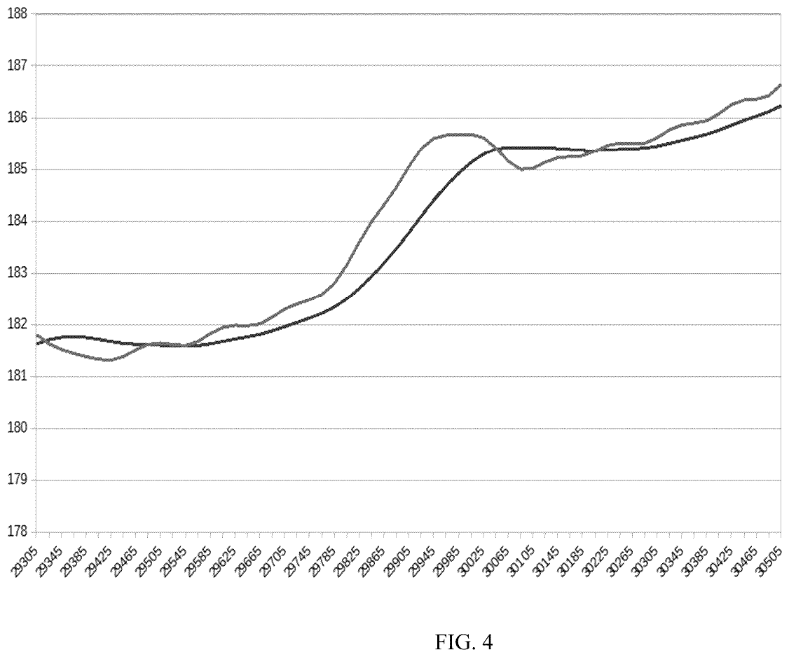

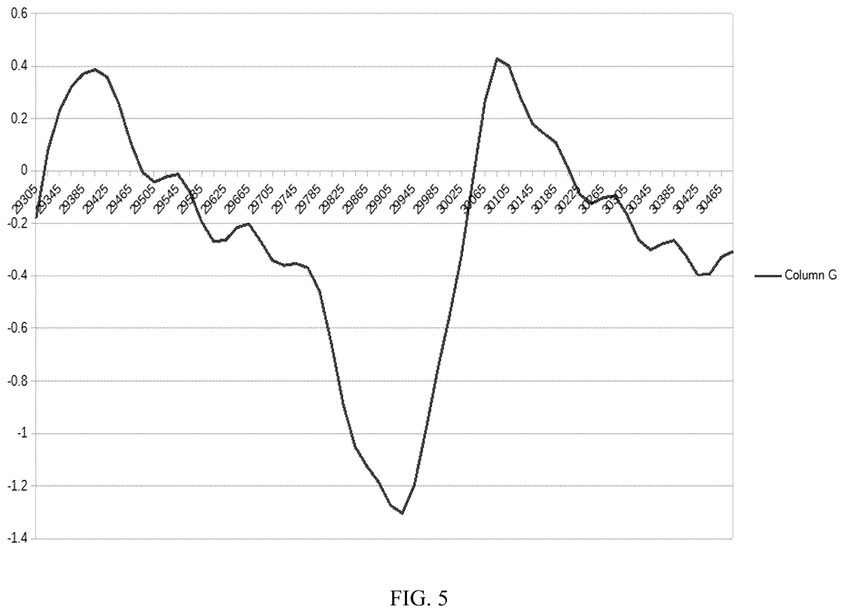

During the pressure measurements of Step 2/304, the resulting data produced by first low pass filter LP1 and second low pass filter LP2 during a single arterial pulse spike (e.g., heartbeat) can be recorded. Example data as graphed lines resulting from the two low pass filters is shown in graph 600 of FIG. 4. FIG. 4 illustrates the collected data resulting from a single arterial pulse spike. The same data is graphed as the data of LP1 minus the data of LP2 as single pulse line 710 in the graph 700 of FIG. 5.

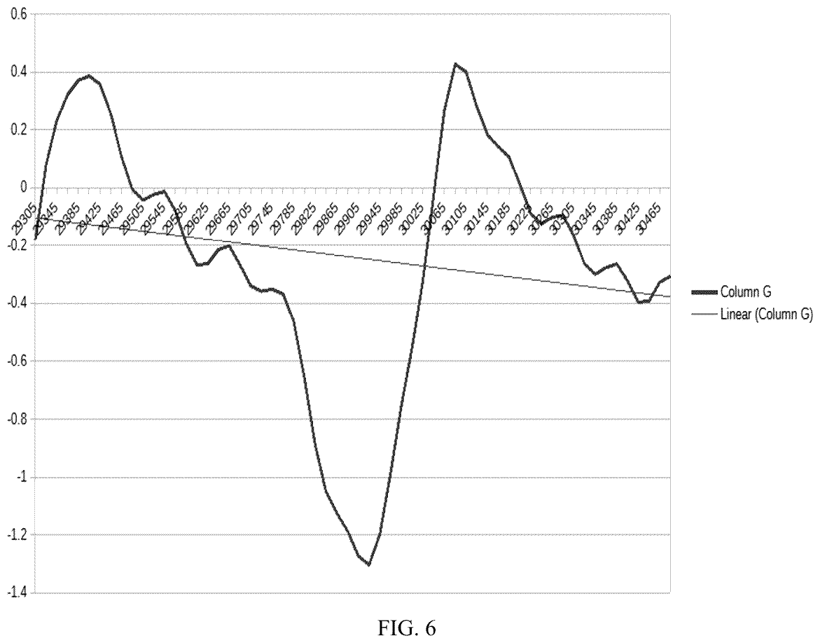

Because pressure measurements are being made as cuff 204 is being inflated and under increasing pressure, method 300 at Step 3/306, compensates for the increasing/decreasing pressure trend by calculating a dynamic linear regression trendline 712, as depicted in FIG. 6. Dynamic linear regression trendline 712 is generated from data from the single pulse line 710.

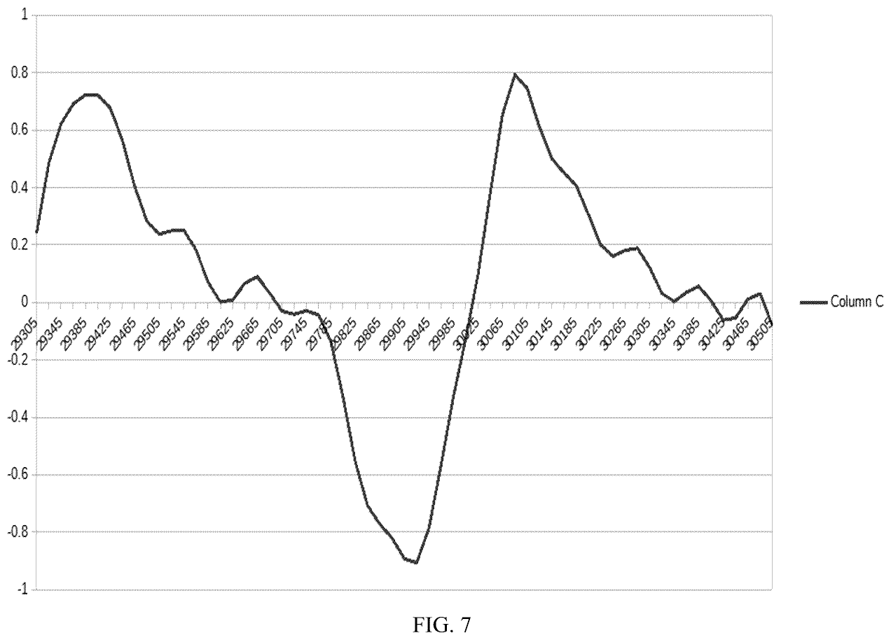

Further at Step 3/306, linear regression trendline 712 is used to generate a modified data set to center or orient single pulse line 710 about the 0 axis value. Such modified data set for single pulse line 710 that has been calculated and graphed is shown in FIG. 7. Single pulse line 710 represents a single arterial pulse spike that was measured/detected by the single arterial pulse spike and can be used in combination with other single pulse lines to determine when LOP is obtained during the inflation of the cuff without having to termination operation of the air pump in air delivery unit 202.

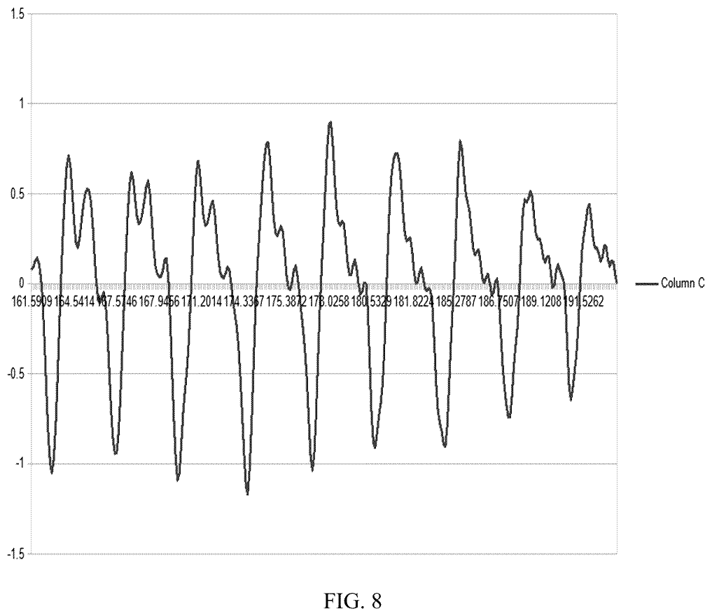

Referring now to FIG. 8, there is shown a graph 800 illustrating a graphed line 810 of a representative sequence of detected arterial pressure spikes (e.g., spikes that are caused by heartbeats). Each of the arterial pressure spikes illustrated in FIG. 8 is a single pulse line that was generated/calculated as discussed above with reference to FIGS. 4-7.

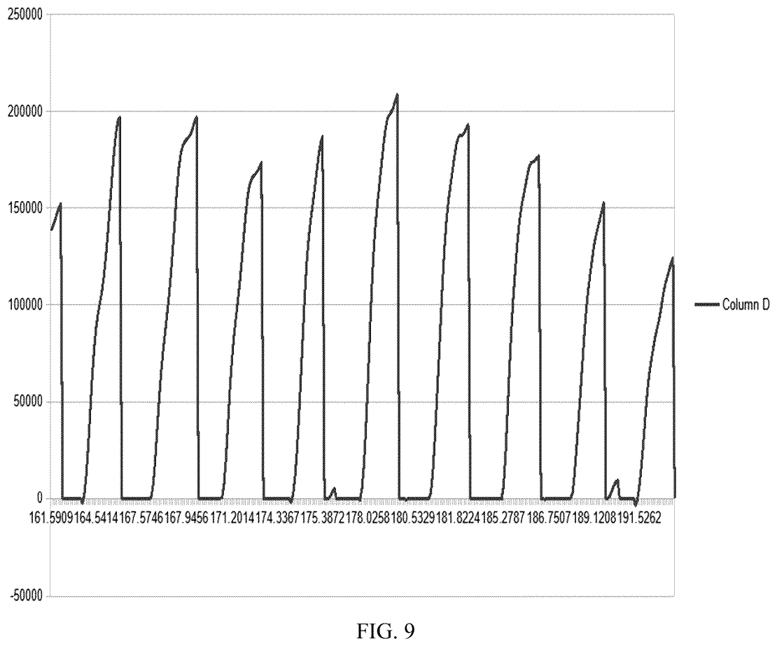

It can be observed in FIG. 8, e.g., in the region generally shown at 812, that the height and/or area of peaks of line 810 denoting peak pressure become lower, narrower and/or have less area under the curve as the pressure and/or pressure pulses in the cuff as sensed by the pressure/pulse sensor reaches and passes LOP due to the increased pressure of the inflating cuff on the artery or arteries. In one non-limiting embodiment of the method 300, at Step 4, 308, the area under each of the peaks of graphed line 810 can be calculated by integrating the pressure points over time for all positive band filter values, as schematically indicated by graphed line 910 of graph 900 shown in FIG. 9.

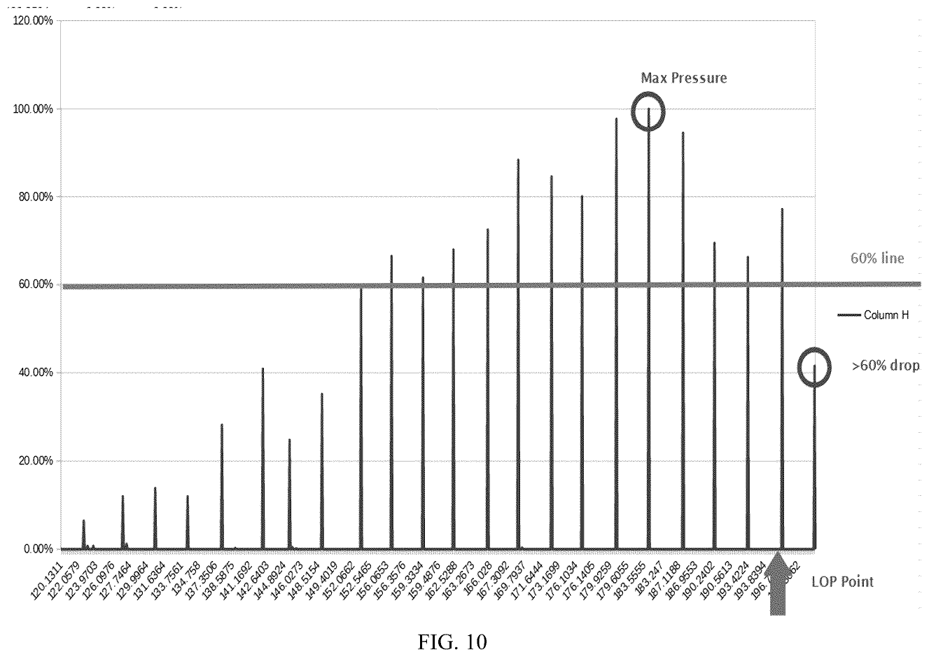

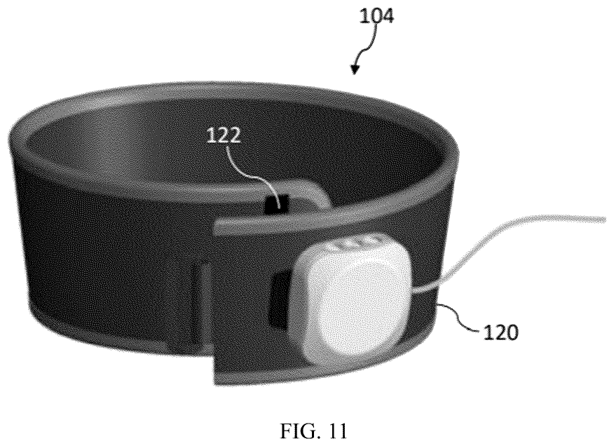

Once the integrated data of pressure pulses is calculated at Step 4/308, at Step 5/310, the LOP can be calculated and further cuff pressurization can stop at the determination that LOP has been obtained. LOP can be calculated by employing a peak detection algorithm to the data gathered in Step 4, 308, to produce the graphed data as shown in graph 1000 shown in FIG. 10. As indicated on graph 1000, each pressure pulse can be graphed to show a maximum pressure for each pulse. Additionally, a maximum pressure 1010 for all the pressure pulses can be measured and reported. Utilizing the peak detection algorithm, a drop in the peak value as a percentage of maximum pressure 1010 can be detected/determined. For example, in one non-limiting embodiment, a drop in the peak value of at least 25% (e.g., 25-100% and all values and ranges therebetween; 60% or greater, etc.), as indicated at line 1012 in FIG. 10, is considered to correspond to the LOP of the cuff. Maximum pressure 1010 can be determined as the maximum detected pressure prior to one or two consecutive measured pressure drops. As indicated in FIG. 10, during the inflation of the cuff, there may be one or two or three subsequent pressure reading that may be less than a prior pressure reading. However, a new maximum pressure reading may later be obtained during cuff inflation. As such, the control system can be configured to not select a maximum pressure until at least two (e.g., 2-20 and all values therebetween) subsequent pressure readings are below the last maximum pressure reading so as to avoid a false or premature maximum pressure value.

Thus, in another non-limiting embodiment, LP1 pressure point at the beginning of the previous heartbeat, shown as peak 1014, is considered to be the LOP point or very close to the LOP point, as the next peak shown as 1016, is below the 60% cut off. As can be appreciated, other percentage drop cut-offs can be used (e.g., 25-100% cut off and all values and ranges therebetween). It is noted that pulses are still present after the obtaining of the LOP due to the air pulsations from the air pump filling the cuff with air.

The method for determining LOP in accordance with the present disclosure is able to account for the pulsations in the cuff caused by the air pump to make a determination as to when LOP occurs in the cuff. The drop in the pulse peak represents the termination of arterial blood flow past the cuff. When blood is flowing past the cuff, the pulses from the blood flow plus the pulsation caused by the flow of air into the cuff from the air pump form the height of the peak of the pulses in the cuff. When blood flow past the cuff is terminated, the pulses caused by the blood flow past the cuff are eliminated, and only the pulses caused by the flow of air into the cuff from the air pump form the height of the pulse peaks; thus, the peaks are reduced.

When utilizing the apparatus and system of the present disclosure, for example, cuff apparatus 100 or cuff apparatus 200 in accordance with method 300 as disclosed herein, a more efficient, precise, and reliable method for determining LOP based on pressure measurements is obtained without the need to stop the operation of the air pump during the detection of pulses in the cuff. It has been found that inflation of a cuff (e.g., 104 or 204) to the LOP can be achieved faster with more precise and reliable results due to one or more of: the reduction in noise due to the predetermined resistance in the air tubes and/or the pulse bottle configuration of the cuff; the low pass filtering of pressure data; calculations of the peak height; area under the peak and/or how wide/narrow the detected/measured heartbeat pulses are; the integration of heartbeat curve to calculate pressure peaks; and the determination of a relative drop as a percentage of the maximum peak pressure. As can be appreciated, the graphs illustrated in FIGS. 4-10 are not required to be generated by cuff apparatus 100 or cuff apparatus 200 and/or a smart device and/or computer that is in wired and/or wireless communication with cuff apparatus 100 or cuff apparatus 200 in accordance with method 300. The data that can be used to form the graphs illustrated in FIGS. 4-10 is generated/calculated by cuff apparatus 100 or cuff apparatus 200 and/or a smart device and/or computer that is in wired and/or wireless communication with cuff apparatus 100 or cuff apparatus 200. Such data can be calculated/determined to determine the LOP in the cuff without the need for generating the one or more graphs illustrated in FIGS. 4-10. However, it can be appreciated that the data that can be used to form the graphs illustrated in FIGS. 4-10 can be optionally displayed by a display on cuff apparatus 100 or cuff apparatus 200 and/or on a display of a smart device and/or computer. Likewise, one or more of the graphs illustrated in FIGS. 4-10 can be optionally displayed by a display on cuff apparatus 100 or cuff apparatus 200 and/or on a display of a smart device and/or computer.

Referring now to FIG. 11, the cuff (e.g., cuff 104) includes an inflatable air system formed of one or more two layers of material. In one non-limiting embodiment, the inflatable air system includes two layers of material coupled together to create one or more air chambers to be inflated with air and/or some other gas or fluid. In one non-limiting configuration, cuff 104 is a single air chamber inflatable cuff. In another non-limiting configuration, cuff 104 includes two or more air chambers. The configuration and shape of the one or more air chambers is non-limiting. The inflatable air system can be configured in cuff 104 such that inflation of the inflatable air system produces compression on a target compression zone that in turn produces a restriction of blood flow in the venous system of a user. Cuff 104 can optionally include an outer material cover 120 having a cavity (not shown) for containing the inflatable air system to partially or fully isolate the inflatable air system from the skin of the user and/or to provide protection to the inflatable air system, and/or to provide structural strength to cuff 104. The inflatable air system can optionally be secured in the cavity to one or more inner surface of outer material cover 120 (e.g., stitching, adhesive, melted connection, etc.). Outer material cover 120 can be generally made of a flexible and durable material (e.g., nylon, Kevlar™, etc.). In one non-limiting embodiment, outer material cover 120 is formed of an inelastic or non-stretch material. Outer material cover 120 can be configured to a) facilitate in distributing the force applied by the inflatable air system to the limb of a user, b) reduce pinching of the user's skin during the inflation of the inflatable air system, and/or c) provide friction between cuff 104 and the user's skin to inhibit or prevent rotation of cuff 104 on the user during use.

Cuff 104 generally includes a connection arrangement to facilitate in the securing of cuff 104 to a user's limb. As illustrated in FIG. 11, cuff 104 can include a “hook and loop” fastener 122 having a loop/loop-like material on the outer surface that is configured to releasably connect to a hook/loop material. A strap arrangement that includes a “hook and loop” fastener can also or alternatively be used to secure the cuff 104 to a user's limb. As can be appreciated, other types of connection arrangements can be used.

Cuff 104 can include one or more input ports such as air input port 110 or fluid sensor port 112 as shown in FIG. 1, and can optionally also include one or more other features (e.g., data/communication input port, power input port, power supply, housing connector for a housing for the air delivery unit and/or the pressure/pulse sensor, pockets, clips, etc.) Air input port 110 is configured to be in fluid communication with the air delivery unit to allow a fluid (e.g., air) to flow into and out of the inflatable air system of the cuff. Fluid sensor port 112 is configured to be in fluid communication with pressure/pulse sensor 206. Air input port 110 and/or fluid sensor port 112 can optionally include a valve component and/or connector (not shown). The location of the one or more input ports on the cuff is non-limiting. Generally, at least one input port is located on an outwardly facing surface of outer material cover 120. One or more of the input ports can be configured to be connected to the air delivery unit (e.g., air pump 102), which can be any of a portable air delivery unit, non-portable air delivery unit, permanently connected air delivery unit, detachably connected air delivery unit, etc. The one or more input ports can optionally include a one-way valve (not shown) to prevent undesired deflation of the air inflatable system. The one or more input ports can optionally be configured to allow for manual deflation of the air inflatable system.

Continuing to refer to FIG. 11, cuff 104 can have a component 124 mounted thereto. In one non-limiting configuration, component 124 is in the formed of a housing that partially or fully includes the pressure/pulse sensor and/or the air delivery unit. The housing can be configured to be connected to an outer surface of outer material cover of the cuff. The cuff can optionally include a housing connector to secure the housing to the cuff. Generally, the housing is configured to be releasably secured to the cuff; however, this is not required. Generally, the housing is configured to be maintained on the cuff during use of the inflated cuff by the user; however, the housing can be configured to be removed from the cuff during use of the cuff by the user. As can be appreciated, component 124 can be located on any portion of the outer material cover. In one non-limiting embodiment, component 124 in the form of a housing that partially or fully includes the pressure/pulse sensor and the air delivery unit is connectable to the cuff so the housing partially or fully overlies air input port 110 and fluid sensor port 112 on the cuff when the housing is connected to the cuff, and wherein the housing and/or the air delivery unit and/or the pressure/pulse sensor include tubing and/or a connector that enables the air delivery unit to be fluidly connected to air input port 110 and the pressure/pulse sensor to be fluidly connected to fluid sensor port 112 when the housing is connected to the cuff. FIG. 11 illustrates a charging/data cable CC connected to the housing via a charging/data port.

In one non-limiting configuration, component 124 in the form of a housing that partially or fully includes the air delivery unit and the pressure/pulse sensor at least partially contains a) a motor; b) a pump; c) optionally a power source; d) one or more pressure sensors and/or one or more pulse sensors; e) one or more circuit boards and other electronics; f) one or more processors (e.g., controller, etc.); g) optionally one or more pressure gauges; h) software; i) optionally one or more displays (e.g., LED screen, OLED screen, etc.); j) optionally a power port; k) optionally a data port (e.g., micro USB port, etc.); l) an optional GPS system; m) optional wireless electronics (e.g., transmitter and/or receiver, etc.) to send/receive wireless signals; n) optionally one or more buttons (e.g., power button, home button, power off button, reset button, etc.); o) optionally one or more selection or scroll buttons to enable manual operation/control of the air delivery unit; p) optionally one or more switch(es) to enable manual operation/control of the air delivery unit; q) connection arrangement to permanently or releasably connect the housing to the cuff; r) optionally one or more air ports; s) optionally one or more valves, t) optionally one or more air tubes, u) optionally one or more temperature sensors, v) optionally a gyroscope, and/or w) optionally one or more lights and/or other indicators to indicate a mode and/or operation and/or status of the air delivery unit and/or pressure/pulse sensor. The shape and size of housing is non-limiting. The material used to form the housing is generally a durable material (e.g., plastic, metal, composite, etc.). In one non-limiting arrangement, the housing is permanently or releasably connected to the cuff and has a size and weight that does not interfere with the use of the cuff while the air delivery unit is connected to the cuff. Generally, the volume of the housing of the air delivery unit is no more than 500 cubic inches, typically 0.5-500 cubic inches (and all values and ranges therebetween), and more typically 1-50 cubic inches. Generally, the weight of the air delivery unit, including housing, is no more than 10 lbs., typically 0.05-10 lbs. (and all values and ranges therebetween), and more typically 0.1-1 lbs.

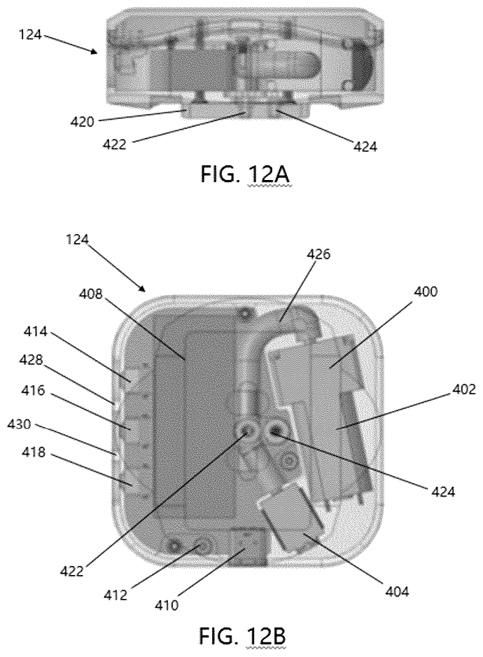

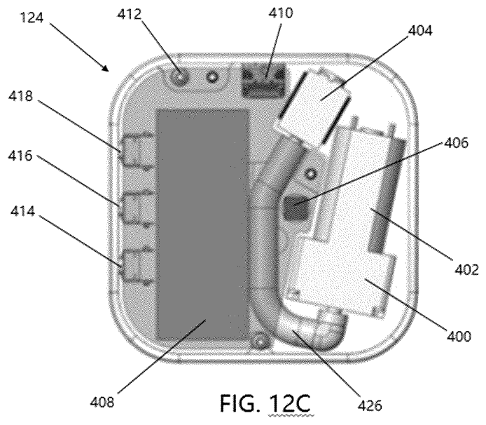

Referring now to FIGS. 12A-C, FIG. 12 illustrates the front, back, and side views of component 124 in the form of a housing that fully includes the air delivery unit and the pressure/pulse sensor. The housing contains a) a motor/pump 400; b) a power source 402; c) a pressure sensor/pulse sensors 404, 406, d) a circuit board/processor 408; e) a power port/data port 410 (e.g., micro USB port, etc.); f) wireless electronics (e.g., transmitter and/or receiver, etc.) 412; g) buttons 414, 416, 418 (e.g., power button, home button, power off button, reset button, etc.); h) connection arrangement 420 to permanently or releasably connect the housing to the cuff; i) air-ports 422, 424; j) air tubes 426, and k) one or more lights 428, 430 and/or other indicators to indicate a mode and/or operation and/or status of the air delivery unit and/or pressure/pulse sensor.

As illustrated in FIGS. 12A-C, a short air tube 426 (e.g. tube that is less than five inches is positioned internally in the housing and forms a fluid connection between motor/pump 400 and air-port 422. Air tube 426 can optionally be connect a pressure sensor 404 used to measure/detect the pressure/pulses in air tube 426. Pressure sensor/pulse sensor 406 is illustrated as being directly connected to air-port 424, thus no tube is used or a tube that is substantially less (e.g., 0.1-30% the length of air tube 426 and all values and ranges therebetween) than the length of air tube 426 is used to fluidly connect pressure sensor/pulse sensor 406 to air-port 424.

The apparatus described herein can optionally have a display that shows the measured pressure in mmHg, the power and/or charge level of the battery in the housing, and optionally displays a warning symbol if some type of warning event has occurred (e.g., battery power level too low, malfunction of air pump, malfunction of electronic/processor of motor/pump unit, maximum measured/detected pressure exceeded, etc.). As can be appreciated, the optional one or more displays can display other or additional material (e.g., selected % LOP, operational information for motor/pump unit and/or LOP pressure, pressure at selected % LOP, operational state or step of the motor/pump unit, e.g., inflating cuff, obtaining limb LOP, deflating cuff to selected % LOP, fully deflating cuff, etc., manual selection of % LOP, selection of arms or legs for cuff inflation, select to fully deflate cuff, time remaining to fully charge the battery, whether the air pump is on/off, etc.).

A system of the present disclosure can include the apparatus described above, as well as suitable operational components to perform the method described above. Further, the system can be configured to be manually controlled by the user activating/pressing/moving buttons, switches, locations on the display, etc., and/or the apparatus can be configured to be controlled wirelessly and/or wired by a computer, tablet, smart phone, and/or other type of smart device. The wired and/or wireless control of the apparatus can be accomplished via a program or app running on a computer, tablet, smart phone, and/or other type of smart device. Wired and/or wireless control of the apparatus can also be accomplished by voice commands. As can be appreciated, the apparatus can include electronics, software, etc. to enable voice control of the apparatus without the need of a computer or other type of smart device. When programs or apps are used to wire and/or wirelessly control the apparatus, these programs or apps can be used to both monitor the operation of the apparatus as well as control the operation of the apparatus.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures or characteristics may be combined in any suitable manner in one or more embodiments.

To aid the Patent Office and any readers of this application and any resulting patent in interpreting the claims appended hereto, Applicant does not intend any of the appended claims or claim elements to invoke 35 U.S.C. 112(f) unless the words “means for” or “step for” are explicitly used in the particular claim.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall there between. The disclosure has been described with reference to the certain embodiments. These and other modifications of the disclosure will be obvious from the disclosure herein, whereby the foregoing descriptive matter is to be interpreted merely as illustrative of the disclosure and not as a limitation. It is intended to include all such modifications and alterations insofar as they come within the scope of the appended claims.

1-34. (canceled)

35. An apparatus for making a calculated determination of when a limb inclusion pressure (LOP) has been obtain in a cuff that is positioned about a user's limb as said cuff is continuously inflated, said apparatus comprising:
said cuff that includes an inflatable bladder; said cuff is configured to encircle the user's limb; said cuff includes a pressure sensor port and an input port; said pressure sensor port and said input port are spaced from one another; both said pressure sensor port and said input port are in fluid communication with said inflatable bladder;
a delivery unit; said delivery unit is configured to provide fluid into said inflatable bladder; said delivery unit is configured to be fluidly connected or interconnected to said inflatable bladder via said input port to provide fluid flow to said inflatable bladder to inflate said inflatable bladder;
a pressure/pulse sensor arrangement; said pressure/pulse sensor arrangement includes a pressure/pulse sensor; said pressure/pulse sensor is configured to measure and/or to detect a pressure and/or a pressure pulse in said inflatable bladder during continuous uninterrupted inflation of said inflatable bladder; said pressure/pulse sensor arrangement is fluidly connected or interconnected to said inflatable bladder via said pressure sensor port; and
a processing arrangement that is configured to make said calculated determination as to when said limb occlusion pressure has been obtained in said inflatable bladder while said cuff is positioned about the user's limb; and
wherein said pressure sensor port and said input port are configured such that a fill air resistance (FAR) of said fluid from said delivery unit to said inflatable bladder is different from a pressure sensor air resistance (PSAR) of said fluid between said inflatable bladder and said pressure/pulse sensor arrangement during inflation of said inflatable bladder.

36. The apparatus as defined in claim 35, wherein said pressure sensor port is spaced from said input port is such that when said cuff encircles the user's limb, a portion of the user's limb is positioned between said pressure sensor port and said input port.

37. The apparatus as defined in claim 35, wherein said input port is configured to allow a fluid to enter said inflatable bladder during inflation of said inflatable bladder; said pressure sensor port is configured to not allow fluid to enter said inflatable bladder during inflation of said inflatable bladder.

38. The apparatus as defined in claim 35, further including a housing; said housing is configured to be connected on an outer surface of said cuff; said housing at least partially includes said delivery unit, said pressure/pulse sensor arrangement, and/or said processing arrangement; said housing is configured to remain connected on said outer surface of said cuff during inflation of said inflatable bladder and use of said cuff by a user while said inflatable bladder is at least partially inflated; said housing partially or fully overlies said input port and/or said pressure sensor port when said housing is connected on an outer surface of said cuff.

39. The apparatus as defined in claim 35, wherein said processing arrangement is configured to perform two or more functions selected from the group consisting of a) calculate a value of pressure spikes or pulses caused by a heartbeat as blood flows through the blood vessel and past said inflated bladder, b) calculate a trend intensity of said pressure spikes or pulses caused by the heartbeat as blood flows through the blood vessel and past said inflated bladder, c) calculate information about an area of a curve of said pressure spikes or pulses caused by the heartbeat as blood flows through the blood vessel and past said inflatable bladder, d) calculate maximum pulsation amplitude of pressure spikes or pulses caused by the heartbeat as blood flows through the blood vessel and past said inflatable bladder, e) calculate when a pulsation amplitude of pressure spikes or pulses caused by the heartbeat as blood flows through the blood vessel and past said inflatable bladder have reduced a predefined amount as compared to said maximum pulsation amplitude of pressure spikes or pulses, f) calculate a frequency of said detected pressure spikes or pulses, g) calculate a maximum pulsation amplitude of said pressure spikes or pulses, h) calculate a width of said pressure spikes or pulses, and/or i) differentiate between pressure spikes or pulses caused by said delivery unit and said pressure spikes or pulses caused by said blood flow through the blood vessel and past said inflatable bladder.

40. The apparatus as defined in claim 39, wherein said processing arrangement is includes first and second low pass filters used to digitally filter said pressure data so as to facilitate in making said determination as to when said limb occlusion pressure has been obtained in said inflatable bladder; said first and second low pass filters have a different cut-off frequency.

41. The apparatus as defined in claim 35, wherein said fill air resistance (FAR) is less than said pressure sensor air resistance (PSAR).

42. The apparatus as defined in claim 35, wherein said fill air resistance (FAR) is greater than said pressure sensor air resistance (PSAR).

43. A system for making a calculated determination of when a limb inclusion pressure (LOP) has been obtain in a cuff that is positioned about a user's limb as said cuff is continuously inflated; said system comprises:
a cuff that includes an inflatable bladder; said cuff is configured to encircle the user's limb; said cuff includes a pressure sensor port and an input port; said pressure sensor port and said input port are spaced from one another; both said pressure sensor port and said input port are in fluid communication with said inflatable bladder;
a delivery unit; said delivery unit is configured to provide fluid into said inflatable bladder; said delivery unit is configured to be fluidly connected or interconnected to said inflatable bladder via said input port to provide fluid flow to said inflatable bladder to inflate said inflatable bladder;
a pressure/pulse sensor arrangement; said pressure/pulse sensor arrangement includes a pressure/pulse sensor; said pressure/pulse sensor is configured to measure and/or detect a pressure and/or a pressure pulse in said inflatable bladder during continuous uninterrupted inflation of said inflatable bladder; said pressure/pulse sensor arrangement is fluidly connected or interconnected to said inflatable bladder via said pressure sensor port; and
a processing arrangement that is configured to make said calculated determination as to when said limb occlusion pressure has been obtained in said inflatable bladder while said cuff is positioned about the user's limb;
wherein said pressure sensor port and said input port are configured such that a fill air resistance (FAR) of said fluid from said delivery unit to said inflatable bladder is different from a pressure sensor air resistance (PSAR) of said fluid between said inflatable bladder and said pressure/pulse sensor arrangement during inflation of said inflatable bladder. (New) The system as defined in claim 43, wherein said pressure sensor port is spaced from said input port is such that when said cuff encircles the user's limb, a portion of the user's limb is positioned between said pressure sensor port and said input port.

45. The system as defined in claim 43, wherein said input port is configured to allow a fluid to enter said inflatable bladder during inflation of said inflatable bladder; said pressure sensor port is configured to not allow fluid to enter said inflatable bladder during inflation of said inflatable bladder.

46. The system as defined in claim 43, further including a housing; said housing is configured to be connected on an outer surface of said cuff; said housing at least partially includes said delivery unit, said pressure/pulse sensor arrangement, and/or said processing arrangement.

47. The system as defined in claim 46, wherein said housing is configured to remain connected on said outer surface of said cuff during inflation of said inflatable bladder and use of said cuff by a user while said inflatable bladder is at least partially inflated; said housing partially or fully overlies said input port and/or said pressure sensor port.

48. The system as defined in claim 43, wherein said processing arrangement is configured to perform two or more functions selected from the group consisting of a) calculate a value of pressure spikes or pulses caused by a heartbeat as blood flows through the blood vessel and past said inflated bladder, b) calculate a trend intensity of said pressure spikes or pulses caused by the heartbeat as blood flows through the blood vessel and past said inflated bladder, c) calculate information about an area of a curve of said pressure spikes or pulses caused by the heartbeat as blood flows through the blood vessel and past said inflatable bladder, d) calculate maximum pulsation amplitude of pressure spikes or pulses caused by the heartbeat as blood flows through the blood vessel and past said inflatable bladder, e) calculate when a pulsation amplitude of pressure spikes or pulses caused by the heartbeat as blood flows through the blood vessel and past said inflatable bladder have reduced a predefined amount as compared to said maximum pulsation amplitude of pressure spikes or pulses, f) calculate a frequency of said detected pressure spikes or pulses, g) calculate a maximum pulsation amplitude of said pressure spikes or pulses, h) calculate a width of said pressure spikes or pulses, and/or i) differentiate between pressure spikes or pulses caused by said delivery unit and said pressure spikes or pulses caused by said blood flow through the blood vessel and past said inflatable bladder.

49. The system as defined in claim 48, wherein said processing arrangement is includes first and second low pass filters used to digitally filter said pressure data so as to facilitate in making said determination as to when said limb occlusion pressure has been obtained in said inflatable bladder; said first and second low pass filters have a different cut-off frequency.

50. The system as defined in claim 43, wherein said fill air resistance (FAR) less than said pressure sensor air resistance (PSAR).

51. The system as defined in claim 43, wherein said fill air resistance (FAR) is greater than said pressure sensor air resistance (PSAR).

52. An apparatus for making a calculated determination of when a limb inclusion pressure (LOP) has been obtain in a cuff that is positioned about a user's limb as said cuff is continuously inflated; said apparatus comprising:
a cuff that includes an inflatable bladder; said cuff is configured to encircle the user's limb; the user's limb is selected from the arm or leg of the user; said cuff includes a pressure sensor port and an input port; said pressure sensor port and said input port are spaced from one another; both said pressure sensor port and said input port are in fluid communication with said inflatable bladder;
a delivery unit; said delivery unit is configured to provide fluid into said inflatable bladder; said delivery unit is configured to be fluidly is connected or interconnected to said inflatable bladder via said input port to provide fluid flow to said inflatable bladder to inflate said inflatable bladder;
a pressure/pulse sensor arrangement; said pressure/pulse sensor arrangement includes a pressure/pulse sensor; said pressure/pulse sensor is configured to generate pressure data by measuring and/or detecting a pressure and/or a pressure pulse in said inflatable bladder during inflation of said inflatable bladder;
a processing arrangement; said processing arrangement is configured to modify said pressure data to calculate when a limb occlusion pressure has been obtained in said inflatable bladder;
a housing; said housing is configured to be connected to an outer surface of said cuff; said housing at least partially includes a) said delivery unit and b) one or both of said pressure/pulse sensor arrangement and said processing arrangement; said housing overlies at least a portion of said pressure sensor port and/or said input port when said housing is connected on an outer surface of said cuff; and
wherein said processing arrangement is configured to perform two or more functions during inflation of said cuff, and wherein said two or more functions are selected from the group consisting of a) calculate a value representative of pressure spikes or pulses caused by a heartbeat as blood flows through a blood vessel and past said inflated bladder while said cuff is positioned about the user's limb, b) calculate a trend intensity of said pressure spikes or pulses caused by the heartbeat as blood flows through the blood vessel and past said inflated bladder while said cuff is positioned about the user's limb, c) calculate information about an area of a curve of said pressure spikes or pulses caused by the heartbeat as blood flows through the blood vessel and past said inflatable bladder while said cuff is positioned about the user's limb, d) calculate maximum pulsation amplitude of pressure spikes or pulses caused by the heartbeat as blood flows through the blood vessel and past said inflatable bladder while said cuff is positioned about the user's limb, e) calculate when a pulsation amplitude of pressure spikes or pulses caused by the heartbeat as blood flows through the blood vessel and past said inflatable bladder have reduced a predefined amount as compared to said maximum pulsation amplitude of pressure spikes or pulses while said cuff is positioned about the user's limb, f) calculate a frequency of said detected pressure spikes or pulses while said cuff is positioned about the user's limb, g) calculate a maximum pulsation amplitude of said pressure spikes or pulses while said cuff is positioned about the user's limb, h) calculated a width of said pressure spikes or pulses while said cuff is positioned about the user's limb, and i) differentiate between pressure spikes or pulses caused by said delivery unit and said pressure spikes or pulses caused by said blood flow through the blood vessel and past said inflatable bladder while said cuff is positioned about the user's limb.

53. The apparatus as defined in claim 52, wherein said processing arrangement is includes first and second low pass filters; said first and second low pass filters are used to digitally filter said pressure data so as to facilitate in making said determination as to when said limb occlusion pressure has been obtained in said inflatable bladder; said first and second low pass filters each have a different cut-off frequency.

54. The apparatus as defined in claim 52, wherein said pressure sensor port and said input port are configured such that a fill resistance (FAR) of said fluid from said delivery unit to said inflatable bladder is different from a pressure sensor air resistance (PSAR) of said fluid between said inflatable bladder and said pressure/pulse sensor arrangement.

55. The apparatus as defined in claim 53, wherein said pressure sensor port and said input port are configured such that a fill air resistance (FAR) of said fluid from said delivery unit to said inflatable bladder is different from a pressure sensor air resistance (PSAR) of said fluid between said inflatable bladder and said pressure/pulse sensor arrangement.

56. The apparatus as defined in claim 54, wherein said fill air resistance (FAR) is greater than said pressure sensor air resistance (PSAR).

57. The apparatus as defined in claim 55, wherein said fill air resistance (FAR) is greater than said pressure sensor air resistance (PSAR).

58. The apparatus as defined in claim 52, wherein said pressure sensor port is spaced from said input port and said spacing is such that when said cuff encircles at least a portion of the user's limb, at least a portion of the user's limb is positioned between said pressure sensor port and said input port.

59. The apparatus as defined in claim 57, wherein said pressure sensor port is spaced from said input port and said spacing is such that when said cuff encircles at least a portion of the user's limb, at least a portion of the user's limb is positioned between said pressure sensor port and said input port.

60. The apparatus as defined in claim 52, wherein said input port is configured to allow a fluid to enter said inflatable bladder during inflation of said inflatable bladder; said pressure sensor port is configured to not allow fluid to enter said inflatable bladder during inflation of said inflatable bladder.

61. The apparatus as defined in claim 59, wherein said input port is configured to allow a fluid to enter said inflatable bladder during inflation of said inflatable bladder; said pressure sensor port is configured to not allow fluid to enter said inflatable bladder during inflation of said inflatable bladder.
